(19) United States
(12) Patent Application Publication
(10) Pub. No.: US 2025/0295913 A1
(43) Pub. Date: Sep. 25, 2025

(54) Using Alternating Electric Fields to Block Pain

(71) Applicant: Novocure GmbH, Baar (CH)

(72) Inventors: Uri WEINBERG, Haifa (IL); Yoram WASSERMAN, Haifa (IL); Mor BEN-TOV KUPERBERG, Haifa (IL)

(73) Assignee: Novocure GmbH, Baar (CH)

(21) Appl. No.: 19/085,909

(22) Filed: Mar. 20, 2025

Related U.S. Application Data
(60) Provisional application No. 63/568,197, filed on Mar. 21, 2024.

(51) Int. Cl. A61N 1/36

(52) U.S. Cl. CPC A61N 1/36021; A61N 1/36034

(57) ABSTRACT

Pain can be blocked in a living subject by positioning a plurality of electrodes at respective locations on the subject's body, and applying an AC signal between the plurality of electrodes. The AC signal has amplitude and frequency characteristics such that, when the AC signal is applied to the electrodes, the AC signal will block pain at a target site. The AC signal has a given frequency between 50 kHz and 1 MHZ, and an amplitude between 75% and 150% of a threshold value. The threshold value is either (a) determined based on a test performed on the subject to determine a lowest amplitude at which the subject experiences electrosensation at the given frequency or (b) based on previously obtained data that specifies a lowest amplitude at which most subjects experience electrosensation at the given frequency.

### CROSS REFERENCE TO RELATED APPLICATIONS

This Application claims the benefit of U.S. Provisional Application 63/568,197, filed Mar. 21, 2024, which is incorporated herein by reference in its entirety.

### BACKGROUND

Hundreds of millions of people experience pain every day, and managing pain can often be a daunting problem. Conventional approaches for managing pain include a wide spectrum of pharmaceuticals (many of which can be dangerous and/or habit-forming), as well as a variety of non-pharmaceutical approaches. But in many cases, the conventional approaches fall short of the desired goal of safely eliminating or ameliorating the subject's pain. There is therefore a strong need for additional approaches for managing pain.

### SUMMARY OF THE INVENTION

One aspect of the invention is directed to a first method for blocking pain experienced by a subject at a target site of a subject's body. The first method comprises positioning a plurality of electrodes at respective locations on the subject's body, and applying a first AC signal between the plurality of electrodes. The respective locations are selected so that when the first AC signal is applied between the plurality of electrodes, the first AC signal will block pain at the target site. The first AC signal has amplitude and frequency characteristics such that when the first AC signal is applied between the plurality of electrodes, the first AC signal will block pain at the target site. The first AC signal has a given frequency between 50 kHz and 1 MHz, and an amplitude between 75% and 150% of a threshold value. The threshold value is either (a) determined based on a test performed on the subject to determine a lowest amplitude at which the subject experiences electrosensation at the given frequency or (b) based on previously obtained data that specifies a lowest amplitude at which most subjects experience electrosensation at the given frequency.

In some instances of the first method, the threshold value is determined based on the test performed on the subject. In some instances of the first method, the threshold value is based on previously obtained data that specifies a lowest amplitude at which at least 80% of subjects experience electrosensation at the given frequency.

In some instances of the first method, prior to the applying, the threshold value is determined by applying a second AC signal to the subject's body at the given frequency; increasing an amplitude of the second AC signal until the subject begins to experience electrosensation; noting the amplitude of the second AC signal at which the subject began to experience electrosensation; and using the noted amplitude as the threshold value.

Optionally, in the instances described in the previous paragraph, the second AC signal is applied to the subject's body subsequent to the positioning using the plurality of electrodes. Optionally, in the instances described in the previous paragraph, the first AC signal has an amplitude between 80% and 99% of the threshold value. Optionally, in the instances described in the previous paragraph, the first AC signal has an amplitude between 100% and 120% of the threshold value.

In some instances of the first method, prior to the applying, the given frequency is determined by applying a second AC signal to the subject's body, wherein the second AC signal is maintained at a particular amplitude; decreasing a frequency of the second AC signal until the subject begins to experience electrosensation; noting the frequency of the second AC signal at which at the subject began to experience electrosensation; and using the noted frequency as the given frequency. In these embodiments, the threshold value corresponds to the particular amplitude of the second AC signal.

Optionally, in the instances described in the previous paragraph, the second AC signal is applied to the subject's body subsequent to the positioning using the plurality of electrodes.

In some instances of the first method, prior to the applying, the given frequency and threshold value are determined by applying a second AC signal to the subject's body; varying the frequency of the second AC signal according to a periodic frequency-varying profile, and increasing an amplitude of the second AC signal while varying the frequency of the second AC signal; upon detecting that the subject has begun experiencing electrosensation, noting the frequency and amplitude of the second AC signal at which at the subject began to experience electrosensation; and using the noted frequency as the given frequency, and using the noted amplitude as the threshold value.

Optionally, in the instances described in the previous paragraph, the periodic frequency-varying profile causes the frequency characteristics of the second AC signal to, during a single periodic cycle of operation, decrease from a maximum frequency to a minimum frequency during a first portion of a periodic cycle, maintain the frequency of the second AC signal at the minimum frequency during a second portion of the periodic cycle, and increase the frequency of the second AC signal to the maximum frequency during a third portion of the periodic cycle.

In some instances of the first method, the locations at which the plurality of electrodes are positioned are selected so the target site is located between the plurality of electrodes. In some instances of the first method, the plurality of electrodes are positioned proximally with respect to the target site. In some instances of the first method, at least one of the electrodes is positioned proximally with respect to the target site, and at least one of the electrodes is positioned distally with respect to the target site.

In some instances of the first method, the first AC signal has a frequency between 90 kHz and 300 kHz. In some instances of the first method, the first AC signal comprises a periodic sequence of bursts of AC voltage, each burst has a duration of less than 50 ms, and the periodic sequence has a period between 100 ms and 2 seconds. In some instances of the first method, the first AC signal comprises a sequence of bursts of AC voltage, each burst has a duration of less than 50 ms, and successive bursts within the sequence are separated by between 100 ms and 2 seconds.

Another aspect of the invention is directed to a first apparatus for blocking pain experienced by a subject at a target site of a subject's body. The first apparatus comprises an AC voltage source and a controller. The AC voltage source is configured to generate a first AC signal. The controller is configured to control the operation of the AC voltage source so that (a) the first AC signal has amplitude and frequency characteristics such that when the first AC signal is applied between a plurality of electrodes positioned at respective locations on the subject's body, the first AC signal will block pain at the target site, (b) the first AC signal has a given frequency between 50 kHz and 1 MHz, and (c) the first AC signal has an amplitude between 75% and 150% of a threshold value. The threshold value is either (a) determined based on a test performed on the subject to determine a lowest amplitude at which the subject experiences electrosensation at the given frequency or (b) based on previously obtained data that specifies a lowest amplitude at which most subjects experience electrosensation at the given frequency.

In some embodiments of the first apparatus, the threshold value is determined based on the test performed on the subject, and the controller is further configured to (a) control the operation of the AC voltage source when performing the test on the subject so that the AC voltage source (i) applies a second AC signal to the subject's body at the given frequency and (ii) increases an amplitude of the second AC signal until the subject begins to experience electrosensation, (b) note the amplitude of the second AC signal at which the subject began to experience electrosensation, and (c) use the noted amplitude as the threshold value.

Optionally, in the embodiments described in the previous paragraph, the amplitude of the first AC signal is set to between 80% and 99% of the threshold value. Optionally, in the embodiments described in the previous paragraph, the amplitude of the first AC signal is set to between 100% and 120% of the threshold value.

In some embodiments of the first apparatus, the first AC signal has a frequency between 90 kHz and 300 kHz. In some embodiments of the first apparatus, the first AC signal comprises a periodic sequence of bursts of AC voltage, wherein each burst has a duration of less than 50 ms, and wherein the periodic sequence has a period between 100 ms and 2 seconds. In some embodiments of the first apparatus, the first AC signal comprises a sequence of bursts of AC voltage, wherein each burst has a duration of less than 50 ms, and wherein successive bursts within the sequence are separated by between 100 ms and 2 seconds.

In some embodiments of the first apparatus, the threshold value is determined based on the test performed on the subject, and the controller is further configured to (a) control the operation of the AC voltage source when performing the test on the subject so that the AC voltage source (i) applies a second AC signal to the subject's body, wherein the second AC signal is maintained at a particular amplitude and (ii) decreases a frequency of the second AC signal until the subject begins to experience electrosensation, (c) note the frequency of the second AC signal at which the subject began to experience electrosensation, and (d) use the noted frequency as the given frequency. In these embodiments, the threshold value corresponds to the particular amplitude of the second AC signal.

In some embodiments of the first apparatus, the threshold value is determined based on the test performed on the subject, and the controller is further configured to (a) control the operation of the AC voltage source when performing the test on the subject so that the AC voltage source (i) applies a second AC signal to the subject's body, (ii) varies the frequency of the second AC signal according to a periodic frequency-varying profile, and (iii) increases an amplitude of the second AC signal while varying the frequency of the second AC signal; upon detecting that the subject has begun experiencing electrosensation, note the frequency and amplitude of the second AC signal at which the subject began to experience electrosensation; and use the noted frequency as the given frequency, and use the noted amplitude as the threshold value.

Optionally, in the embodiments described in the previous paragraph, the periodic frequency-varying profile causes the frequency characteristics of the second AC signal to, during a single periodic cycle of operation, decrease from a maximum frequency to a minimum frequency during a first portion of a periodic cycle, maintain the frequency of the second AC signal at the minimum frequency during a second portion of the periodic cycle, and increase the frequency of the second AC signal to the maximum frequency during a third portion of the periodic cycle.

In some embodiments of the first apparatus, the threshold value is based on previously obtained data that specifies a lowest amplitude at which at least 80% of subjects experience electrosensation at the given frequency.

Another aspect of the invention is directed to a second method for blocking pain experienced by a subject at a target site of a subject's body. The second method comprises positioning a plurality of electrodes at respective locations on the subject's body, and applying a first AC signal between the plurality of electrodes. The respective locations are selected so that when the first AC signal is applied between the plurality of electrodes, the first AC signal will block pain at the target site. The first AC signal has amplitude and frequency characteristics such that when the first AC signal is applied between the plurality of electrodes, the first AC signal will block pain at the target site. And the first AC signal has a given frequency between 50 kHz and 1 MHz.

In some instances of the second method, the first AC signal has an amplitude of at least 30 V peak to peak. In some instances of the second method, the first AC signal has an amplitude of 30-90 V peak to peak.

In some instances of the second method, the amplitude of the first AC signal is set to at least 25% of a threshold value, and the threshold value is determined based on a test performed on the subject to find a lowest amplitude at which the subject experiences electrosensation at the given frequency.

In some instances of the second method, the amplitude of the first AC signal is set to 50-100% of a threshold value, and the threshold value is determined based on a test performed on the subject to find a lowest amplitude at which the subject experiences electrosensation at the given frequency.

Optionally, in the instances described in the previous paragraph, prior to the applying, the threshold value is determined by applying a second AC signal to the subject's body at the given frequency; increasing an amplitude of the second AC signal until the subject begins to experience electrosensation; noting the amplitude of the second AC signal at which the subject began to experience electrosensation; and using the noted amplitude as the threshold value. Optionally, in these instances, the second AC signal is applied to the subject's body subsequent to the positioning using the plurality of electrodes.

In some instances of the second method, the amplitude of the first AC signal is set to 50-100% of a threshold value, the threshold value is determined based on a test performed on the subject to find a lowest amplitude at which the subject experiences electrosensation at the given frequency, and the first AC signal has an amplitude between 80% and 99% of the threshold value.

In some instances of the second method, prior to the applying, the given frequency is determined by: applying a second AC signal to the subject's body, wherein the second AC signal is maintained at a particular amplitude; decreasing a frequency of the second AC signal until the subject begins to experience electrosensation; noting the frequency of the second AC signal at which at the subject began to experience electrosensation; and using the noted frequency as the given frequency. Optionally, in these instances, the second AC signal is applied to the subject's body subsequent to the positioning using the plurality of electrodes.

In some instances of the second method, the locations at which the plurality of electrodes are positioned are selected so the target site is located between the plurality of electrodes. In some instances of the second method, the plurality of electrodes are positioned proximally with respect to the target site. In some instances of the second method, at least one of the electrodes is positioned proximally with respect to the target site, and at least one of the electrodes is positioned distally with respect to the target site.

In some instances of the second method, the first AC signal has a frequency between 90 kHz and 300 kHz. In some instances of the second method, the first AC signal comprises a sequence of bursts of AC voltage, each burst has a duration of less than 50 ms, and successive bursts within the sequence are separated by between 100 ms and 2 seconds.

Another aspect of the invention is directed to a second apparatus for blocking pain experienced by a subject at a target site of a subject's body. The second apparatus comprises an AC voltage source and a controller. The AC voltage source is configured to generate a first AC signal. The controller is configured to control the operation of the AC voltage source so that (a) the first AC signal has amplitude and frequency characteristics such that when the first AC signal is applied between a plurality of electrodes positioned at respective locations on the subject's body, the first AC signal will block pain at the target site, (b) the first AC signal has a given frequency between 50 kHz and 1 MHz, and (c) the first AC signal has an amplitude between 50% and 100% of a threshold value. The threshold value is determined based on a test performed on the subject to find a lowest amplitude at which the subject experiences electrosensation at the given frequency.

In some embodiments of the second apparatus, the controller is further configured to perform the test by (a) controlling the operation of the AC voltage source so that the AC voltage source (i) applies a second AC signal to the subject's body at the given frequency and (ii) increases an amplitude of the second AC signal until the subject begins to experience electrosensation; (b) noting the amplitude of the second AC signal at which the subject began to experience electrosensation; and (c) using the noted amplitude as the threshold value. Optionally, in these embodiments, the amplitude of the first AC signal is set to between 80% and 99% of the threshold value.

In some embodiments of the second apparatus, the first AC signal has a frequency between 90 kHz and 300 kHz. In some embodiments of the second apparatus, the first AC signal comprises a sequence of bursts of AC voltage, each burst has a duration of less than 50 ms, and successive bursts within the sequence are separated by between 100 ms and 2 seconds.

In some embodiments of the second apparatus, the controller is further configured to perform the test by (a) controlling the operation of the AC voltage source when performing the test on the subject so that the AC voltage source (i) applies a second AC signal to the subject's body, wherein the second AC signal is maintained at a particular amplitude and (ii) decreases a frequency of the second AC signal until the subject begins to experience electrosensation; (b) noting the frequency of the second AC signal at which the subject began to experience electrosensation; and (c) using the noted frequency as the given frequency. In these embodiments, the threshold value corresponds to the particular amplitude of the second AC signal.

### BRIEF DESCRIPTION OF THE DRAWINGS

FIG. 1A depicts an apparatus for blocking pain that includes electrodes positioned on a subject's arm and an AC source that applies AC signals to the electrodes.

FIG. 1B depicts bands to secure the electrodes of FIG. 1A to the arm of the subject.

FIG. 2 depicts the periodic frequency variations in one example of an AC signal that can provide a pain blocking effect.

FIG. 3 is a graph illustrating voltage levels of an AC signal at a given frequency that is applied to a subject as a sequence of several bursts.

FIG. 4 is a flowchart of an example pain blocking procedure.

Various embodiments are described in detail below with reference to the accompanying drawings, wherein like reference numerals represent like elements.

### DESCRIPTION OF THE PREFERRED EMBODIMENTS

This application is directed to systems, apparatuses, methods, and other implementations to apply electric fields to a region in the subject's body in order to achieve a pain blocking effect for pain that the subject experiences as emanating from an area (referred to as the “target site” or “pain area”) on the subject's body. Note that the area to which the electric field is applied and the pain area/target site do not necessarily overlap.

Tumor Treating Fields (TTFields) therapy is a proven approach for treating tumors using alternating electric fields at frequencies e.g., between 50 kHz-1 MHz, more commonly 100-300 kHz. The alternating electric fields are induced by electrode assemblies (e.g., arrays of capacitively coupled electrodes, also called transducer arrays) placed on the subject's skin on opposite sides of a region of the subject's body. When an AC voltage is applied between opposing electrode assemblies, an alternating electric field is formed in that region.

When treating a subject using alternating electric fields, the efficacy of treatment is strongly dependent on the amount of time that the alternating electric fields are applied. For example, when TTFields are used to treat a tumor, it is preferable to apply the alternating electric fields for at least 14 hours a day, and preferably even longer. In addition, higher amplitudes are strongly associated with higher efficacy of treatment. However, as the amplitude of the alternating electric field increases, and/or as the frequency of the alternating electric field decreases (e.g., to the vicinity of 100 kHz), some subjects experience an unpleasant sensation referred to herein as “electrosensation.” This electrosensation could be, for example, a vibratory sensation, paresthesia, and/or a twitching or contraction sensation of muscle fibers. The inventors believe that the electrosensation originates from interactions between the alternating electric fields and nerve cells (i.e., neurons) that are positioned near or adjacent to the electrode assemblies.

In the context of treating a subject using alternating electric fields (e.g., TTFields), electrosensation is a problem because it can cause some subjects to reduce the amount of time that the alternating electric fields are applied each day (which will reduce the efficacy of the treatment). And in extreme cases, the electrosensation could even cause some subjects to discontinue their treatment. But the inventors have discovered a beneficial side effect that is related to electrosensation. More specifically, at or around the combination of amplitude and frequency at which electrosensation occurs, pain experienced by the subject at certain areas of their body can be blocked or at least partially alleviated. And in many cases, the pain blocking effect will occur at significantly lower amplitudes. In some embodiments, this pain blocking effect is achieved by intentionally causing electrosensation. In other embodiments, the pain blocking effect can be achieved without causing electrosensation by setting the amplitude to a lower level (e.g., 80%, 50%, or even 25% of the amplitude that will trigger electrosensation in the subject).

Some embodiments block pain experienced by a subject at a target site of a subject's body. These embodiments include an AC voltage source configured to generate a first AC signal, and a controller configured to control the operation of the AC voltage source so that (a) the first AC signal has amplitude and frequency characteristics such that when the first AC signal is applied between a plurality of electrodes positioned at respective locations on the subject's body, the first AC signal will block pain at the target site, (b) the first AC signal has a given frequency between 50 kHz and 1 MHz, and (c) the first AC signal has an amplitude between 75% and 150% of a threshold value. The threshold value is either (a) determined based on a test performed on the subject to determine a lowest amplitude at which the subject experiences electrosensation at the given frequency or (b) based on previously obtained data that specifies a lowest amplitude at which most subjects experience electrosensation at the given frequency.

Some embodiments block pain experienced by a subject at a target site of a subject's body. These embodiments include an AC voltage source configured to generate a first AC signal, and a controller configured to control the operation of the AC voltage source so that (a) the first AC signal has amplitude and frequency characteristics such that when the first AC signal is applied between a plurality of electrodes positioned at respective locations on the subject's body, the first AC signal will block pain at the target site, (b) the first AC signal has a given frequency between 50 kHz and 1 MHz, and (c) the first AC signal has an amplitude between 50% and 100% of a threshold value. The threshold value in these embodiments is determined based on a test performed on the subject to find a lowest amplitude at which the subject experiences electrosensation at the given frequency.

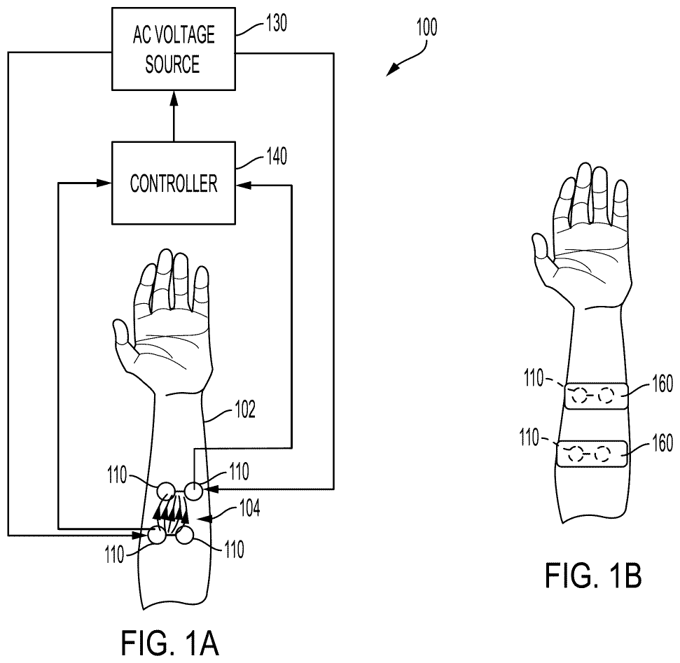

FIG. 1A depicts an apparatus 100 for blocking pain experienced by a subject. The apparatus 100 includes a plurality of electrodes 110 positioned at respective locations on a forearm 102 of a subject. Note that the electrodes 110 can be positioned at other locations on the body, generally in proximity to the area where the subject is experiencing pain. The electrodes 110 can be deployed to surround the area from which the subject is experiencing pain, so that the target site 104 (where the subject is experiencing pain) is located between the electrodes 110. For example, at least one of the electrodes 110 can be positioned proximally with respect to the target site 104, and at least one of the electrodes 110 can be positioned distally with respect to the target site, as depicted in FIG. 1A. But note that the deployment of electrodes can be such that the area where electrical currents are applied does not necessarily overlap the painful area. For example, in FIG. 1A the electrodes are positioned on the subject's forearm, but the application of alternating currents through those electrodes to impose electrical fields (with electrical characteristics determined in the manner more particularly described below) can, in some situations, block or alleviate pain at another part of the arm that is located distally beyond the electrodes (e.g., the wrist).

A wide variety of electrodes can be used in the implementations described herein, including but not limited to the electrode assemblies described in US 2023/0043071, entitled “Electrode Assembly for Applying Tumor Treating Fields (TTFields) that Include a Sheet of Graphite,” US 2021/0402179, entitled “Flexible Transducer Arrays with a Polymer Insulating Layer for Applying Tumor Treating Fields (TTFields),” US 2021/0402179, entitled “Flexible Transducer Arrays with a Polymer Insulating Layer for Applying Tumor Treating Fields (TTFields),” and U.S. Pat. No. 8,715,203, entitled “Composite electrode.” Each of these documents is incorporated herein by reference in its entirety.

The electrodes 110 positioned on the subject's body part (e.g., the subject's arm, leg, back, etc.) can be secured to the skin using a conductive adhesive, such as conductive hydrogel adhesive or a conductive non-hydrogel polymer adhesive. To further secure the electrodes to the skin and maintain their positions on the subject's body part, further electrode-securing mechanisms can be used. For example, FIG. 1B shows securing bands 160 wrapped over the electrodes 110 (the electrodes 110, which would ordinarily not be visible through the bands 160, are shown in broken lines). The bands 160 can be manufactured from stretchable/flexible materials that can be used with body parts of different dimensions and contours, and may also be structured to operate to press the electrodes (or electrode assemblies) against the subject's body. Such stretchable/flexible materials may include breathable materials, e.g., cotton fabrics, through which sweat can permeate. The stretchable/flexible bands 160 can be re-usable bands, in which case they can include fasteners (not shown) such as hook-and-loop fasteners (e.g., Velcro®) attached to opposite ends of the bands to allow adjustable fastening of the bands to form a closed loop to surround different dimensioned body parts. Other types of fastening mechanisms can be used, including various adhesive materials, belt-and-buckle fasteners, etc. Alternatively, the bands 160 of FIG. 1B can be disposable bands such as bandages (e.g., band-aid type bandages) with a front surface (the surface contacting a subject's skin) at least partly covered with an adhesive layer so as to affix the bandage to the body part.

Returning to FIG. 1A, the apparatus 100 further includes an AC voltage source 130 configured to generate and apply AC signals to the electrodes 110, which imposes alternating electric fields in a region 104 between the electrodes 110. Note that while FIGS. 1A and 1B depict two electrodes 110 above the region 104 and two electrodes 110 below the region 104, the number of electrodes on each side of the region 104 can vary (e.g., between 1 and 20). When the electric characteristics of the generated AC signal are adapted to particular values (e.g., a pre-determined threshold voltage and a particular frequency), the imposed electric field will result in a pain blocking effect for pain emanating from an area (the “pain area” or “pain site”) of the subject. (As noted above, the pain area is not necessarily within the region 104 in which the electric field is formed). The AC voltage source 130 is configured to apply an AC voltage with a frequency between 50 kHz and 1 MHZ, with voltage levels that will typically be on the order of 50-150 VRMS or 20-150 VRMS.

In the embodiment depicted in FIG. 1A, the AC voltage source 130, and the apparatus 100 in general, is controlled by a controller 140 (e.g., a processor-based controller). The controller 140 controls characteristics of the voltage delivered by the AC voltage source 130 (including amplitude and frequency, and optionally other characteristics such as waveform and duty cycle). This control can be based on previously determined electrical characteristics for a particular subject that result, when applied to the particular subject (and possibly applied at a particular location using a particular arrangement of electrodes) in the blocking of pain experienced by the subject. As will be discussed in greater detail below, the controller 140 can also be configured to control the AC source voltage 130 while performing a test to determine electrical characteristics of AC signals (e.g., lowest amplitude, at a given frequency, at which the subject experiences electrosensation) that would help block or reduce pain experienced by the subject.

In some embodiments, the controller 140 controls operation of the AC voltage source (including electrical characteristics of the generated AC signals) based further on operating conditions. For example, the controller 140 can be in electrical communication with various sensors deployed in the system 100, and receive measurement data from such sensors. For instance, the controller 140 can use temperature measurements (e.g., measured by thermistors) to adjust the AC signal that is applied to the electrodes 110 in order to maintain their temperatures below a safety threshold (e.g., 41° C.). This can be accomplished, for example, by measuring temperatures of the electrodes, and controlling the AC signals generated by the AC voltage source 130 based on the measured temperatures (e.g., adjusting the amplitude of the AC signal or suspending the application of the AC signal for a period of time).

In the example depicted in FIG. 1A, the electrodes are arranged with one pair of electrodes 110 positioned above the region 104 and another pair of electrodes 110 positioned below the region 104. All of the electrodes within any given pair are electrically coupled to each other (and will therefore be at the same electrical potential). Voltage applied from the AC voltage source 130 between the upper electrodes 110 and the lower electrodes 110 results in the formation of an electric field between those two sets of electrodes 110. The electrical characteristics (e.g., voltage level, frequency, duty cycles, waveform) of the AC signal generated by the AC voltage source 130, will form an associated electrical field that, for a particular individual subject, blocks or reduces pain experienced by the subject. Thus, for example, for a particular individual subject, electrical characteristics for the AC voltage signal that were previously determined for that subject as being able to block or reduce the pain experienced by the subject (e.g., based on performing a test for the particular subject) can be used to set the apparatus for pain blocking treatment. Alternatively, if the electrical characteristics of the AC voltage that would result in the formation of an electric field to block or reduce the pain experienced by the subject are not yet known, the electrodes can be arranged in the same configuration (or a different configuration) as that shown in FIG. 1A, and the particular electric characteristics that result in pain-blocking electric field are determined (e.g., in the manner discussed below).

The apparatus 100 can generate AC signals with electrical characteristics that have been previously determined for a particular individual based on performing a test (i.e., at an earlier time) for that subject. In this situation, the controller 140 retrieves (e.g., from local memory or from a remote server) data associated with that particular individual that specifies a voltage and a frequency to which the AC signals should be set to achieve pain blocking for the individual. The controller 140 (which can be part of the AC voltage source 130) instructs the AC voltage source to produce signals with the specified amplitude and frequency. The retrieved data for a subject may optionally also include information about the particular configuration/arrangement of electrodes that, when the AC signal is applied to the electrodes (at the particular voltage and the given frequency) will result in a pain blocking effect for pain experienced by the subject.

When the electrical characteristics of the AC signal that would block or reduce the pain for a given subject are not known, the apparatus 100 can perform a test to determine those characteristics. In some embodiments, this test is performed immediately before the AC signal is applied to the subject, using the same hardware that will ultimately be used to apply the pain-blocking AC signal. Alternatively, the test can be performed at any prior time (e.g., minutes, hours, days, weeks, or months earlier) using either the same hardware that will ultimately be used to apply the pain-blocking AC signal or using different hardware.

Once the test is performed, and the electrical characteristics have been established, pain management therapy for the subject can proceed. Optionally, the electrical characteristic values determined through performance of the test can be recorded (e.g., locally in a memory device of the controller 140, or at a remote server). And during a subsequent pain treatment for the particular subject (generally for the same type of pain for which the test was conducted), the recorded electrical characteristics can be retrieved and used to configure the AC voltage source 130. As noted above, the data recorded following performance of the test may also include electrode information, e.g., number and type of electrodes, and configuration information representative of how the electrodes are arranged on the body of the subject.

A variety of approaches can be used to determine the electrical characteristics of the AC signals that are applied to the electrodes 110 to achieve a pain blocking or reducing effect. In a first example approach (also referred to as the amplitude sweep approach), a test is performed on the subject to determine a threshold value, representing the lowest amplitude for an AC signal, for a given frequency, at which the subject experiences electrosensation. For instance, a plurality of electrodes are positioned at respective location on the subject's body. Typically, the electrodes are placed in the vicinity of the site on the body from which the subject is experiencing pain. In some situations, the electrodes can be placed (according to an arrangement which can be similar to that depicted in FIG. 1A) so that the target site 104 (where the subject is experiencing pain) is located between the electrodes 110 so that when an electric field is imposed upon activation of the AC voltage source, the electric field will pass through the target site. For example, at least one of the electrodes 110 can be positioned proximally with respect to the target site 104, and at least one of the electrodes 110 can be positioned distally with respect to the target site, as depicted in FIG. 1A. In other situations, the electrodes can all be positioned proximally with respect to the target site (which means that the electrodes will be positioned between the target site and the spinal cord). This type of arrangement might be needed when it is otherwise difficult to place the target site between electrodes, for example when the target site is at a joint (e.g., an elbow or wrist).

After the electrodes have been placed, a given frequency between 50 kHz and 1 MHz for the AC signal is set. The AC voltage source is set to a starting amplitude (typically one that is sufficiently low that it is unlikely to cause the subject to experience electrosensation) and is activated. The voltage level is then gradually increased (e.g., by increasing the amplitude by a pre-determined step size at pre-determined time periods). Eventually, after the amplitude level has been sufficiently raised, the subject will start experiencing electrosensation (e.g., manifested as a vibratory sensation, paresthesia, and/or a twitching or contraction sensation of muscle fibers). The onset of the electrosensation can be reported by the subject, or can be detected using specialized sensors to monitor muscle behavior, and to determine the onset of the electrosensation in the body of the subject. Once electrosensation has been experienced or detected, the amplitude of the AC signal is noted, and that amplitude is then used to represent the threshold value (i.e., the lowest amplitude that causes electrosensation for a given frequency of the AC signal). Thus, in some embodiments, the controller 140 can be configured to control the operation of the AC voltage source when performing the test on the subject so that the AC voltage source (i) applies a test AC signal between the plurality of electrodes at the given frequency and (ii) increases the amplitude of the test AC signal until the subject begins to experience electrosensation. The controller is additionally configured to note the amplitude of the test AC signal at which the subject began to experience electrosensation, and use the noted amplitude as the threshold value.

Having determined the threshold voltage, for the given frequency (and/or other characteristics of the AC signals and apparatus setup, such as the electrode configuration), the apparatus can be set to commence applying AC signal to the electrodes on the subject's body to block or reduce pain at the target site. The actual amplitude of the AC signal to be applied once the pain blocking therapy is to commence (i.e., after having determined the threshold value) can be between 75% to 150% of the determined threshold voltage value in some embodiments. In other embodiments it can be 25-150%, 25-120%, 25-100%, 25-99%, 33-150%, 33-120%, 33-100%, 33-99%, 40-150%, 40-120%, 40-100%, 40-99%, 50-150%, 50-120%, 50-100%, 50-99%, 60-150%, 60-120%, 60-100%, 60-99%, 70-150%, 70-120%, 70-100%, 70-99%, 80-150%, 80-120%, 80-100%, or 80 -99% of the determined threshold voltage value.

In some embodiments, the AC signal is set to an amplitude between 100% and 120% of the threshold value. And although this amplitude will result in electrosensation, many subjects will prefer a mild version of electrosensation to the pain that they are trying to avoid.

For many subjects, the pain blocking effect occurs *prior* to the onset of the electrosensation effect, i.e., prior to reaching the determined threshold voltage value. For these subjects, the amplitude of the AC signal can be set to 80-99% of the threshold value, or to 25-99%, 25-99%, 33-99%, 40-99%, 50-99%, 60-99%, or 70-99% of the threshold value. Note that the lower limit of the percentage range that provides a pain blocking effect may vary from subject to subject. The percentage value selected may also depend on such factors as the severity of the pain experienced by the subject, the desired length of the treatment, etc. Alternatively, the amplitude of the AC signal can be set to a default percentage, e.g., between 80% to 99%, of the threshold value, regardless of the presence or absence of certain conditions or factors.

Another example approach for determining electrical characteristics values for the AC signal is by determining an AC signal frequency, for a particular AC signal amplitude level, that causes the subject to experience electrosensation. It has been observed that when lowering the AC signal frequency, there is a point where the lowered frequency of AC signal causes the onset of electrosensation. Thus, the AC signal amplitude can be set to a particular amplitude level (e.g., an amplitude level between 50-150 VRMS or 20-150 VRMS), and the frequency at which electrosensation is triggered in the subject is then determined. To determine this electrosensation trigger frequency, the plurality electrodes (or electrode assemblies) are positioned on the subject (here too, some pre-determined electrode configuration can be used). A test AC signal is then applied between the plurality of electrodes, with the test AC signal being maintained at the particular amplitude. The frequency of the AC signal starts out at an initial value and is gradually decreased (e.g., by a pre-determined step size, and at pre-determined time periods) until the subject begins to experience electrosensation (alternatively, sensors can be used to detect the onset of electrosensation). Once the subject has experienced the electrosensation effect, the frequency at which the electrosensation effect began is noted, and that noted frequency is used as the given frequency when the actual therapeutic application of an AC signal commences.

Accordingly, under the latter approach, the controller 140 may further be configured to control the operation of the AC voltage source when performing the test on the subject so that the AC voltage source (i) applies a test AC signal between the plurality of electrodes, with the test AC signal being maintained at a particular amplitude and (ii) decreases a frequency of the second AC signal until the subject begins to experience electrosensation. The controller 140 is additionally configured to note the frequency of the second AC signal at which the subject began to experience electrosensation, and use the noted frequency as the given frequency. Here the threshold value corresponds to the particular amplitude that the test AC signal was set to at the beginning of the test.

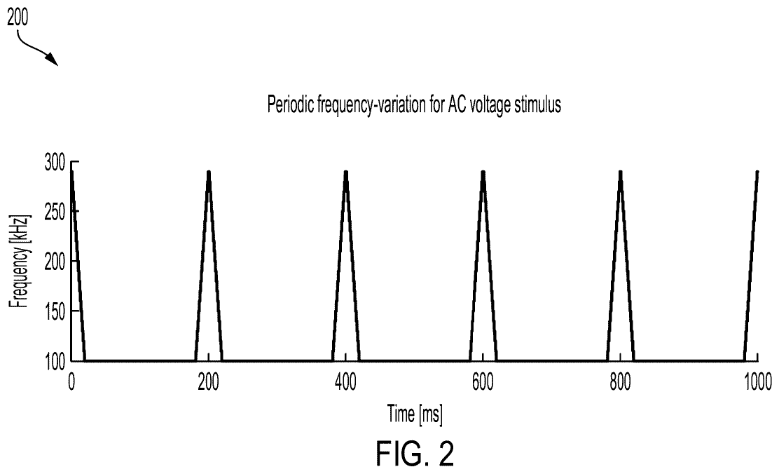

Another approach for determining electrical characteristics for the AC signal when used to block or reduce pain is a hybrid approach in which both the frequency of the AC signal and its amplitude are varied. FIG. 2 includes a graph 200 illustrating periodic frequency variations for an AC voltage stimulus. The frequency varying profile shown in FIG. 2 was used in an experiment with the same apparatus depicted in FIG. 1A. In the experiment, two pairs of ceramic discs 110 were placed on an arm of a subject about 6 cm apart from each other. The frequency profile of the AC signal, as shown in the graph 200, operated at a base frequency of 100 kHz, with periodic excursions every 200 mS up to 300 kHz and back down again to 100 kHz. Each excursion lasted 40 mS. While the frequency was being cycled in this manner, the amplitude was increased from a starting voltage level until the subject felt an electrosensation.

An experiment was performed to verify that a subject who is experiencing electrosensation did not feel pain. This was performed by pinching the arm of the subject with tweezers or a toothpick. In the particular experiment that was conducted, the subject reported the feeling of touch, but not pain.

Accordingly, in this example, the controller 140 is configured to cause the frequency characteristics of a test AC signal to decrease from a maximum frequency to a minimum frequency during a first portion of a periodic cycle, to maintain the frequency of the second AC signal at the minimum frequency (e.g., 100 kHz) during a second portion of the periodic cycle, and increase the frequency of the test AC signal to the maximum frequency during a third portion of the periodic cycle. During the periodic cycle, the amplitude of the AC signal is slowly increased (e.g., by steps of 0.5V per periodic cycle).

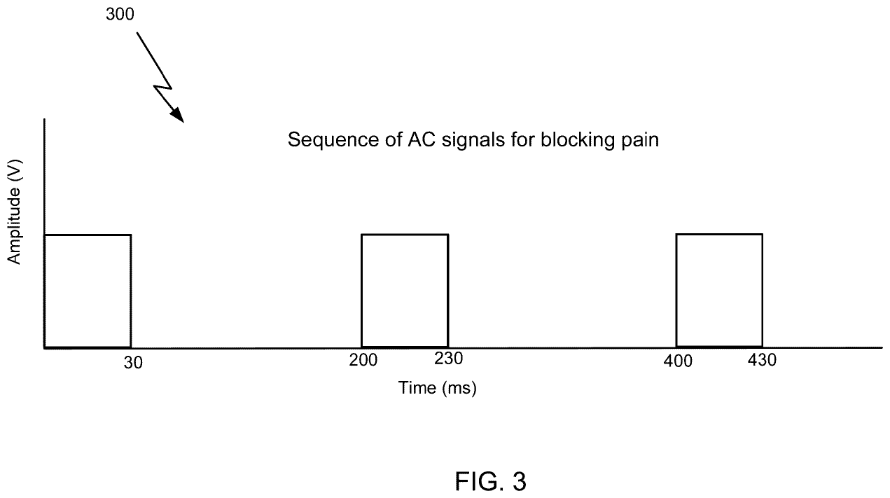

In some embodiments, the AC voltage source is configured (e.g., based on control signals received from the controller 140) to apply the AC signal (e.g., at a voltage determined according to the testing approaches described above) as a sequence of bursts that can be periodic or non-periodic. For example, the graph 300 depicted in FIG. 3 illustrates voltage levels of an AC signal (at a given frequency) that is applied to a subject as a sequence of several bursts. In the example depicted in FIG. 3, a 30 mS burst of AC signal occurs every 200 ms (resulting in a duty cycle of 15%).

In some embodiments, the AC signal includes a periodic sequence of bursts of AC with each burst having a duration of less than 50 mS. In some embodiments, the periodic sequence of bursts has a period of between 100 ms and 2 seconds. Note that a sequence of bursts can be time-limited (e.g., 60-300 seconds). Applying a sequence of bursts for a time-limited period avoids generating excess heat at the site of the electrodes. In other embodiments (not shown), the sequence of bursts can be non-periodic. In these embodiments, the duration of some bursts, or the duration of the respective periods/intervals of those bursts, is unequal.

The pain-blocking effect of the alternating electric field can persist for some time after the application of the AC signal to the electrode has stopped. Accordingly, applying a periodic waveform such as the one illustrated in FIG. 3 can achieve the pain blocking effect, while also reducing adverse effects of heat generation at the electrode sites, and/or reducing the overall effective duration (i.e., the total time duration at which non-zero voltage is applied to the electrodes) at which the subject is receiving the treatment.

As noted above, in various embodiments, the controller 140 can be configured to control operation of the AC voltage source 130 in response to changes in operating conditions, such as a rise in the temperature. Thus, the controller can be configured to adjust the burst sequence to, for example, increase the period (e.g., from 200 ms to 400 ms), shorten the burst sizes (e.g., from 50 ms to 30 ms), or shorten a sequence length if, for example, sensors coupled to the electrodes indicate a rise in temperature. The controller 140 can also be configured to monitor other conditions (e.g., based on biometric measurements from the subject, or based on operating conditions of the apparatus 100), and make necessary adjustments to the characteristics of the sequence of bursts.

As noted, the amplitude and frequency of the AC signal that is applied to block pain experienced by a particular subject can be determined based on a test performed on the subject during which either the frequency or the amplitude (or both) of the AC signal produced by the AC voltage source 130 are swept until the subject begins to experience electrosensation. These electrical characteristics can be determined for a particular arrangement of electrodes, and for a particular chosen waveform. That is, different electrode configurations (e.g., different number of electrodes, different types of electrodes, different locations of the electrodes on the subject's body, different spatial arrangements, etc.), and/or different waveforms may result in a different frequency (for a particular amplitude level) or in a different amplitude (for a given frequency) at which the subject would begin to experience electrosensation.

Therefore, upon identifying the amplitude and frequency to be used to block pain, the electrode configuration and waveform characteristics that were used to identify the amplitude and frequency of the AC signal can be noted and recorded. Subsequently, at some future point when the subject needs to undergo another pain blocking treatment session, in addition to setting the AC voltage source to generate an AC signal with the previously determined amplitude and frequency, the electrode configuration that was previously used with the previously determined amplitude, frequency, and/or waveform can be replicated.

The approaches discussed above are based on finding appropriate electrical characteristics of an AC signal for a particular subject. Another approach that can be used to perform pain blocking therapy for subjects is one that is based on data regarding the use of the apparatus 100 of FIG. 1 on a population of subjects.

Such general performance information may include the number of subjects that experienced electrosensation at various combinations of amplitude and frequency (and optionally for particular waveforms and/or electrode configurations). From this data, a determination can be made about the electrical characteristics that are likely to trigger electrosensation for most subjects. For example, the value at which 80% of subjects experienced electrosensation at a given frequency can be determined. That value can then be used as the threshold value discussed above. Optionally, if data that accounts for the demographic characteristics of the subject (e.g., sex, age, height, weight, etc.) is available, the characteristics of the AC signal can be set based on a more limited set of data that corresponds to the universe of subjects that match the subject being treated.

Yet another approach that can be used to perform pain blocking therapy for subjects is for the apparatus 100 to use a predetermined voltage threshold e.g., between 20 and 100 Volts peak to peak for all subjects. For example, the apparatus 100 can use a predetermined voltage threshold of 20-25, 25-30, 30-40, 40-50, 50-60, 60-70, 70-80, 80-90, 90-100, 30-60, 30-90, 40-80, 40-90, or 45-90 Volts peak to peak for all subjects. The apparatus 100 was tested on a limited number of volunteers, and the ranges of 30-60, 40-80, and 45-90 V peak to peak were found to provide a noticeable pain blocking effect for respective test subjects.

While the various pain blocking approaches described herein were discussed in relation to the onset of electrosensation in a subject (to determine appropriate AC electrical characteristics values for blocking pain), in some embodiments, other physiological effects can be used as a way of determining pain blocking AC signal characteristics. An example of one such physiological effect is the occurrence of an action potential activity. During certain types of electrical stimulation of biological tissue, the electrically evoked compound action potential (ECAP) represents the approximately synchronous firing of a population of electrically stimulated nerve fibers. Upon the application of an electrical signal of sufficient energy to activate nerve fibers, fibers of different diameters and in different locations are activated at roughly the same time (e.g., within fractions of milliseconds) and their action potentials (APs) propagate at different velocities to the vicinity of a recording electrode. Further, different nerve fibers of different diameters, which have different activation thresholds and conduction velocities, convey different signals, e.g., of types of sensation (vibration, temperatures, hair movement, muscle contraction-joint position, etc.).

ECAP associated with electrosensation can be measured using a set of electrodes positioned on a subject's skin. These electrodes detect the compounded sum of the individual APs arriving at approximately the same time, which appear as a curve of a given amplitude and duration. ECAP measuring electrodes can be positioned near the electric field generating electrodes (e.g., the electrodes 110 of FIG. 1A) used to block pain. For example, a passive electrode (to detect electric activity) can be placed near the upper electrodes 110 of FIG. 1A, while another passive electrode can be placed close to the lower electrodes 110. Signals from the ECAP measuring electrodes (which can be, e.g., on the order of mV) are forwarded to an ECAP measurement system (not shown). The ECAP measurement system processes those signals (e.g., using an amplifier and an analog to digital converter). The processed signals can then be used to determine a likelihood that the subject is experiencing electrosensation and/or is close to a threshold beyond which electrosensation is expected. This determination can be based on lookup tables indicating likely occurrence of electrosensation based on the detected values of ECAP activity, using a machine learning system to predict likelihood of electrosensation, or using any other approach relating the ECAP activity to the onset of electrosensation. Further details regarding ECAP and measurement thereof are provided, for example, in US 2023/0414955, entitled “Closed-Loop Technique to Reduce Electrosensation While Treating a Subject Using Alternating Electric Fields,” which is incorporated herein by reference in its entirety.

ECAP measurements can be combined with the amplitude sweep approach or the frequency sweep approach described above. For example, in the amplitude sweep approach, the AC signal is set to a given frequency (e.g., between 100 kHz and 500 kHz), and the amplitude is set to an initial value and is gradually increased. During the amplitude sweep, the ECAP measuring electrodes deployed near the pain-blocking electrodes passively measure electrical activity, and forward the measurements to the controller 140. The controller 140 determines, based on the received measurements whether electrosensation has been triggered or is about to be triggered. Once the controller 140 determines that electrosensation has been triggered or is about to be triggered, the value of the amplitude of the AC signal that has been gradually increased is noted. That noted value represents the threshold value, based on which the pain blocking AC signal electrical characteristics are set.

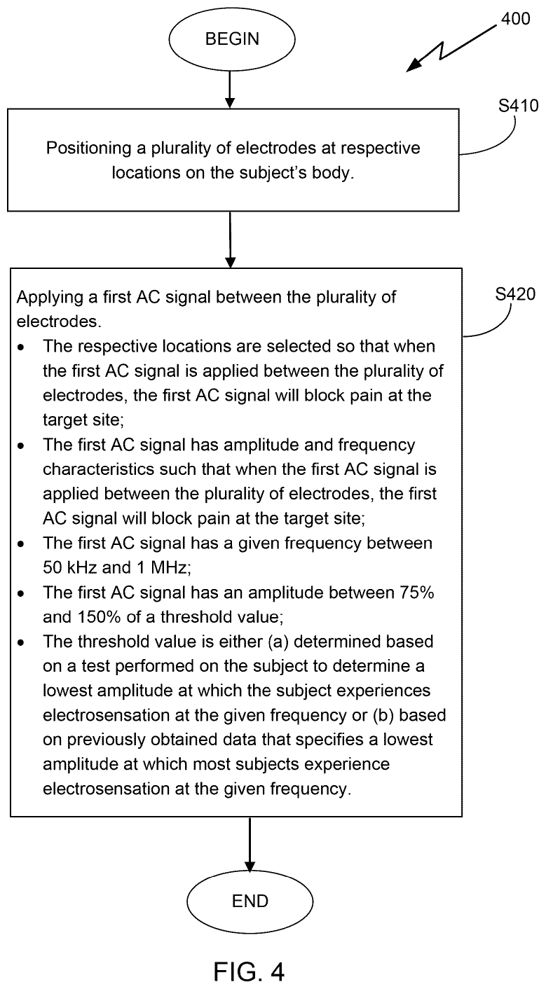

FIG. 4 is a flowchart of an example procedure 400 for blocking pain experienced by a subject at a target site of a subject's body. The procedure includes positioning (at S410) a plurality of electrodes at respective locations on the subject's body, and applying (at S420) a first AC signal between the plurality of electrodes. Under the procedure 400, the respective locations are selected so that when the first AC signal is applied between the plurality of electrodes, the first AC signal will block pain at the target site. Further, the first AC signal has amplitude and frequency characteristics such that when the first AC signal is applied between the plurality of electrodes, the first AC signal will block pain at the target site. Also, the first AC signal has a given frequency between 50 kHz and 1 MHz, the first AC signal has an amplitude between 75% and 150% of a threshold value, and the threshold value is either (a) determined based on a test performed on the subject to determine a lowest amplitude at which the subject experiences electrosensation at the given frequency or (b) based on previously obtained data that specifies a lowest amplitude at which most subjects experience electrosensation at the given frequency.

In some embodiments, prior to the applying, the threshold value is determined by applying a second AC signal (also referred to as a “test AC signal”) to the subject's body at the given frequency, increasing an amplitude of the second AC signal until the subject begins to experience electrosensation, noting the amplitude of the second AC signal at which the subject began to experience electrosensation, and using the noted amplitude as the threshold value. In some embodiments, the first AC signal has an amplitude between 80% and 99% of the threshold value. In other embodiments, the first AC signal has an amplitude between 100% and 120% of the threshold value.

In some embodiments, a test is performed to find a frequency that triggers electrosensation in the subject. In such embodiments, prior to the applying, the given frequency is determined by applying a second AC signal to the subject's body, with the second AC signal being maintained at a particular amplitude, decreasing a frequency of the second AC signal until the subject begins to experience electrosensation, noting the frequency of the second AC signal at which the subject began to experience electrosensation, and using the noted frequency as the given frequency. In these embodiments, the threshold value will be the particular amplitude of the second AC signal.

In some embodiments, a test is performed to find a combination of amplitude and frequency that triggers electrosensation in the subject. In such embodiments, prior to the applying, the given frequency and threshold value is determined by applying a second AC signal to the subject's body, varying the frequency of the second AC signal according to a periodic frequency-varying profile, and increasing an amplitude of the second AC signal while varying the frequency of the second AC signal. Upon detecting that the subject has begun experiencing electrosensation, noting the frequency and amplitude of the second AC signal at which the subject began to experience electrosensation, and using the noted frequency as the given frequency, and the noted amplitude as the threshold value. In some embodiments, the periodic frequency-varying profile may cause the frequency characteristics of the second AC signal to, during a single periodic cycle of operation, decrease from a maximum frequency to a minimum frequency during a first portion of a periodic cycle, maintain the frequency of the second AC signal at the minimum frequency during a second portion of the periodic cycle, and increase the frequency of the second AC signal to the maximum frequency during a third portion of the periodic cycle.

In some embodiments, the locations for the plurality of electrodes are selected so the target site is located between the plurality of electrodes. In other embodiments, the plurality of electrodes are positioned proximally with respect to the target site. In some embodiments, the first AC signal has a frequency between 90 kHz and 300 kHz.

In some embodiments, the first AC signal includes a periodic sequence of bursts of AC voltage, with each burst having a duration of less than 50 ms, and the periodic sequence has a period between 100 ms and 2 seconds. In some embodiments, the first AC signal includes a sequence of bursts of AC voltage, with each burst having a duration of less than 50 ms, and with successive bursts within the sequence being separated by between 100 ms and 2 seconds. In some embodiments, the threshold value is based on previously obtained data that specifies a lowest amplitude at which at least 80% of subjects experience electrosensation at the given frequency.

Headings are provided for convenience only and are not to be construed to limit the invention in any manner. Embodiments illustrated under any heading or in any portion of the disclosure can be combined with embodiments illustrated under the same or any other heading or other portion of the disclosure. Any combination of the elements described herein in all possible variations thereof is encompassed by the invention unless otherwise indicated herein or otherwise clearly contradicted by context.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method for blocking pain experienced by a subject at a target site of a subject's body, the method comprising:
positioning a plurality of electrodes at respective locations on the subject's body; and
applying a first AC signal between the plurality of electrodes,
wherein the respective locations are selected so that when the first AC signal is applied between the plurality of electrodes, the first AC signal will block pain at the target site,
wherein the first AC signal has amplitude and frequency characteristics such that when the first AC signal is applied between the plurality of electrodes, the first AC signal will block pain at the target site, and
wherein the first AC signal has a given frequency between 50 kHz and 1 MHz.

2. The method of claim 1, wherein the first AC signal has an amplitude of at least 30 V peak to peak.

3. The method of claim 1, wherein the first AC signal has an amplitude of 30-90 V peak to peak.

4. The method of claim 1, wherein the amplitude of the first AC signal is set to at least 25% of a threshold value, and
wherein the threshold value is determined based on a test performed on the subject to find a lowest amplitude at which the subject experiences electrosensation at the given frequency.

5. The method of claim 1, wherein the amplitude of the first AC signal is set to 50-100% of a threshold value, and
wherein the threshold value is determined based on a test performed on the subject to find a lowest amplitude at which the subject experiences electrosensation at the given frequency.

6. The method of claim 5, wherein, prior to the applying, the threshold value is determined by:
applying a second AC signal to the subject's body at the given frequency;
increasing an amplitude of the second AC signal until the subject begins to experience electrosensation;
noting the amplitude of the second AC signal at which the subject began to experience electrosensation; and
using the noted amplitude as the threshold value.

7. The method of claim 6, wherein the second AC signal is applied to the subject's body subsequent to the positioning using the plurality of electrodes.

8. The method of claim 5, wherein the first AC signal has an amplitude between 80% and 99% of the threshold value.

9. The method of claim 1, wherein, prior to the applying, the given frequency is determined by:
applying a second AC signal to the subject's body, wherein the second AC signal is maintained at a particular amplitude;
decreasing a frequency of the second AC signal until the subject begins to experience electrosensation;
noting the frequency of the second AC signal at which at the subject began to experience electrosensation; and
using the noted frequency as the given frequency.

10. The method of claim 9, wherein the second AC signal is applied to the subject's body subsequent to the positioning using the plurality of electrodes.

11. The method of claim 1, wherein the locations at which the plurality of electrodes are positioned are selected so the target site is located between the plurality of electrodes.

12. The method of claim 1, wherein the plurality of electrodes are positioned proximally with respect to the target site.

13. The method of claim 1, wherein at least one of the electrodes is positioned proximally with respect to the target site, and at least one of the electrodes is positioned distally with respect to the target site.

14. The method of claim 1, wherein the first AC signal has a frequency between 90 kHz and 300 kHz.

15. An apparatus for blocking pain experienced by a subject at a target site of a subject's body, the apparatus comprising:
an AC voltage source configured to generate a first AC signal; and
a controller configured to control the operation of the AC voltage source so that (a) the first AC signal has amplitude and frequency characteristics such that when the first AC signal is applied between a plurality of electrodes positioned at respective locations on the subject's body, the first AC signal will block pain at the target site, (b) the first AC signal has a given frequency between 50 kHz and 1 MHz, and (c) the first AC signal has an amplitude between 50% and 100% of a threshold value,
wherein the threshold value is determined based on a test performed on the subject to find a lowest amplitude at which the subject experiences electrosensation at the given frequency.

16. The apparatus of claim 15, wherein the controller is further configured to perform the test by
(a) controlling the operation of the AC voltage source so that the AC voltage source (i) applies a second AC signal to the subject's body at the given frequency and (ii) increases an amplitude of the second AC signal until the subject begins to experience electrosensation,
(b) noting the amplitude of the second AC signal at which the subject began to experience electrosensation, and
(c) using the noted amplitude as the threshold value.

17. The apparatus of claim 16, wherein the amplitude of the first AC signal is set to between 80% and 99% of the threshold value.

18. The apparatus of claim 15, wherein the first AC signal has a frequency between 90 kHz and 300 kHz.

19. The apparatus of claim 15, wherein the first AC signal comprises a sequence of bursts of AC voltage, wherein each burst has a duration of less than 50 ms, and wherein successive bursts within the sequence are separated by between 100 ms and 2 seconds.

20. The apparatus of claim 15, wherein the controller is further configured to perform the test by
(a) controlling the operation of the AC voltage source when performing the test on the subject so that the AC voltage source (i) applies a second AC signal to the subject's body, wherein the second AC signal is maintained at a particular amplitude and (ii) decreases a frequency of the second AC signal until the subject begins to experience electrosensation,
(b) noting the frequency of the second AC signal at which the subject began to experience electrosensation, and
(c) using the noted frequency as the given frequency,
wherein the threshold value corresponds to the particular amplitude of the second AC signal.
